(19) United States
(12) Patent Application Publication
(10) Pub. No.: US 2026/0295098 A1
(43) Pub. Date: Oct. 1, 2026

(54) COMPOUNDS USEFUL FOR IN VIVO IMAGING OF PROTEIN OXIDATION AND/OR CANCER TREATMENT

(71) Applicant: Wake Forest University Health Sciences, Winston-Salem, NC (US)

(72) Inventors: Cristina M. Furdui, Clemmons, NC (US); Leslie B. Poole, Winston-Salem, NC (US); S. Bruce King, Walnut Cove, NC (US)

(21) Appl. No.: 19/691,664

(22) Filed: May 29, 2026

Related U.S. Application Data
(62) Division of application No. 17/260,245, filed on Jul. 17, 2019, now abandoned.
(60) Provisional application No. 62/699,207, filed on Jul. 17, 2018.

(51) Int. Cl. A61K 51/04; A61P 35/00; C07B 59/00; C07C 235/40; C07D 249/04

(52) U.S. Cl. CPC A61K 51/0453; A61P 35/00; C07B 59/002; C07C 235/40; C07D 249/04; C07B 2200/05

(57) ABSTRACT

Provided herein are compounds of Formula I, Formula II Formula III which are useful in labeling tissues in a subject (e.g., PET imaging of a subject), for treatment of cancer, and/or for preparing a medicament. Also provided are compounds containing a group that is reactive with sulfenylated proteins for treatment of cancer and/or for preparing a medicament. Methods of synthesis of the compounds and precursor compounds are also provided.

### CROSS-REFERENCE TO RELATED APPLICATIONS

This application is a divisional application of U.S. application Ser. No. 17/260,245 filed Jan. 14, 2021, which is a 35 U.S.C. § 371 national phase entry of PCT Application PCT/US2019/042114, filed Jul. 17, 2019, and published in English on Jan. 23, 2020, as International Publication No. WO 2020/018613, and which claims the benefit of U.S. Provisional Patent Application Ser. No. 62/699,207, filed Jul. 17, 2018, the disclosure of each of which is incorporated by reference herein in its entirety.

### BACKGROUND

Positron-emission tomography (PET) is an imaging technique that detects the signal of a positron-emitting radionuclide or “tracer” administered to a subject. A commonly-used tracer is fluorodeoxyglucose (FDG), having the tracer fluorine-18 (¹⁸F). This glucose analog is taken up by cells, and tissues with high glucose uptake such as cancer cells or tissues can be labeled. However, new PET tracers for use in imaging are needed, in particular to aid in selection of cancer patients most likely to benefit from radiation and other cancer therapies, thus avoiding unproductive exposure and informing treatment decisions to enhance the response to treatment.

Because the radionuclides used in PET scanning have short half-lives (for example, ¹⁸F has a half-life of around 110 minutes), they must be used rapidly after the tracers are produced, which is typically achieved using a cyclotron in close proximity to the PET imaging facility. This limits the ability to use the radionuclides at early steps during synthesis to produce tracers prior to administration.

### SUMMARY

Provided herein according to some embodiments is a compound of Formula I:

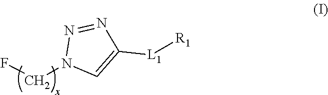

wherein:
   F is fluoro (e.g., ¹⁸F);
   x is an integer of from 1 to 10;
   L₁ is a linker; and
   R₁ is a group that is reactive with sulfenylated proteins,
or a pharmaceutically acceptable salt thereof.

Also provided is a compound of Formula II or Formula III:

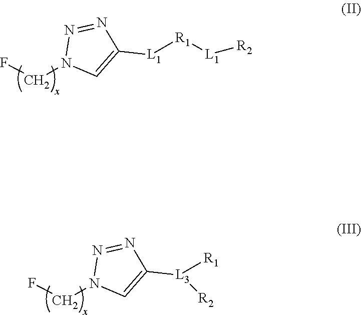

wherein:
   F is fluoro (e.g., ¹⁸F);
   x is an integer of from 1 to 10;
   L₁ is a first linker;
   R₁ is a group that is reactive with sulfenylated proteins;
   L₂ is a second linker;
   L₃ is a branched linker; and
   R₂ is a cellular directing group (e.g., fructose, folic acid, or diethyl amino coumarin), or a pharmaceutically acceptable salt thereof.

In some embodiments of the compounds, L₁ comprises a C1-C10 alkyl, a ketone, an amide, an ester, a carbamate, a urea, an ether, a carbonate, or a combination thereof.

In some embodiments, R₁ comprises a 1,3 dicarbonyl group or a bicyclononyne.

In some embodiments, R₁ is selected from the group consisting of:

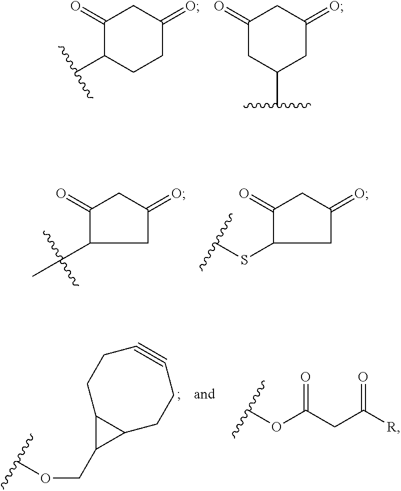

wherein R is an alkyl, ester or amide group.

In some embodiments, R₁ is selected from the group consisting of:

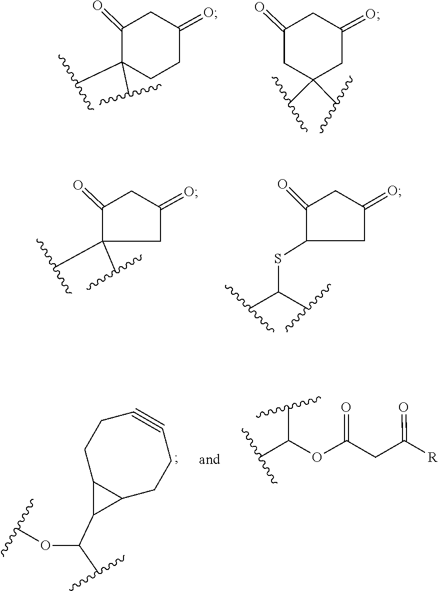

wherein R is an alkyl, ester or amide group.

In some embodiments, the compound is selected from the group consisting of:

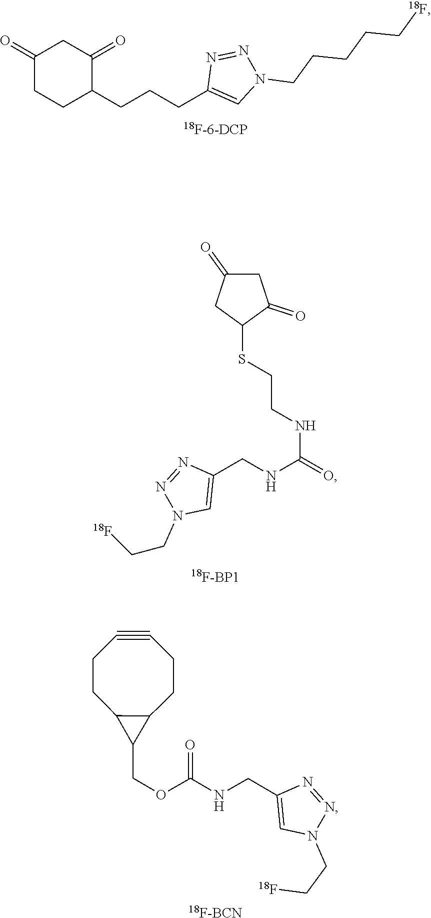

and pharmaceutically acceptable salts thereof.

Also provided is a method of synthesizing a compound taught herein, comprising:
reacting a compound of formula A: N₃—(CH₂)ₓ₊₁—Z, wherein Z is a leaving group (e.g., tosylate or mesylate), and x is as defined above, with fluorine (e.g., ¹⁸F) to form a compound: N₃—(CH₂)ₓ₊₁—F;
providing an alkyne-containing compound of formula B:

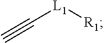

and then
reacting the compound N₃—(CH₂)ₓ₊₁—F with the alkyne-containing compound of formula B, said reacting carried out by a copper-catalyzed Click reaction, to thereby synthesize the compound.

Further provided is a method of synthesizing a compound taught herein, comprising:
reacting a compound of formula A: N₃—(CH₂)ₓ₊₁—Z, wherein Z is a leaving group (e.g., tosylate or mesylate), and x is as defined above, with an alkyne-containing compound of formula B:

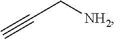

said reacting carried out by a copper-catalyzed Click reaction, to form a compound C:

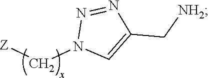

and then
adding L₁-R₁ to the compound to form a compound D (e.g., through amide bond formation of activated carboxylic acids):

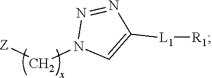

and then
reacting the compound D with fluorine to displace Z with the fluorine (e.g., ¹⁸F), to thereby synthesize the compound.

Also provided is a method of labeling tissues in a subject, comprising: administering a compound taught herein to said subject, and then performing a PET scan on the subject, wherein the PET scan detects the presence or absence of binding of said compound to said tissues, the presence of binding indicating the presence of sulfenylated proteins in said tissues.

In some embodiments, the labeling is carried out during cancer imaging, neuroimaging (e.g., Alzheimer's), cardiology imaging (e.g., hibernating myocardium, atherosclerosis, ischemia/reperfusion injury), infectious disease imaging (e.g., infection-induced inflammatory response), or imaging measuring musculoskeletal activity.

In some embodiments, the tissues comprise cancerous tissues.

In some embodiments, the administering is carried out by parenteral administration.

Further provided is a method for determining whether a cancerous tissue has an increased likelihood of responding to radiation treatment, comprising: administering a compound taught herein to said subject, and then performing a PET scan on the subject, wherein the PET scan detects the presence or absence of binding of said compound to said cancerous tissue, the presence of binding indicating an increased likelihood of response of said cancerous tissue to radiation treatment.

Also provided is a method of treatment for a cancer in a subject in need thereof, comprising administering to the subject a treatment-effective amount of a compound: L₁-R₁, or L₁-R₁-L₂-R₂, wherein: L₁ and L₂ are each independently present or absent, and when present is independently a C1-C10 alkyl, a ketone, an amide, an ester, a carbamate, a urea, an ether (e.g., polyethylene glycol), a carbonate, or a combination of two or more thereof, R₁ is a group that is reactive with sulfenylated proteins; and R₂ is a cellular directing group (e.g., fructose, folic acid, or diethyl amino coumarin), or a pharmaceutically acceptable salt thereof.

In some embodiments, the treatment-effective amount is from 1, 2, 5 or 10 to 20, 30, 40 or 50 mg per kg; or from 0.08, 0.16, 0.4, or 0.81 to 1.6, 2.4, 3.2 or 4 mg/kg.

In some embodiments, the administering is carried out by parenteral administration.

In some embodiments, the method further includes administering radiation therapy to the subject.

In some embodiments, the cancer is determined to be resistant to radiation treatment or treatment targeting receptor tyrosine kinases (e.g., by PET imaging with a compound as taught herein).

In some embodiments, the cancer is determined to be resistant to treatment with an epidermal growth factor receptor (EGFR) antagonist (e.g., erlotinib, imatinib, afatinib, etc.).

Also provided is a compound of the formula: L₁-R₁, or L₁-R₁-L₂-R₂, wherein: L₁ and L₂ are each independently present or absent, and when present is independently a C1-C10 alkyl, a ketone, an amide, an ester, a carbamate, a urea, an ether (e.g., polyethylene glycol), a carbonate, or a combination of two or more thereof,
R₁ is a group that is reactive with sulfenylated proteins; and
R₂ is a cellular directing group (e.g., fructose, folic acid, or diethyl amino coumarin),
or a pharmaceutically acceptable salt thereof.

Further provided are precursor compounds useful to make the above-noted compounds. In some embodiments, the precursor compound is a compound of Formula D:

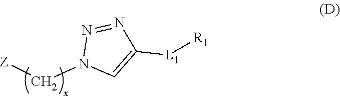

wherein:
   Z is a leaving group;
   x is an integer of from 1 to 10;
   L₁ is a linker; and
   R₁ is a group that is reactive with sulfenylated proteins.

Also provided is a precursor compound E or F:

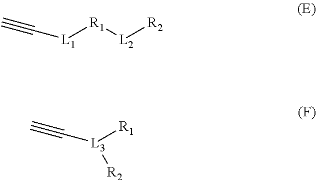

wherein:
   L₁ is a first linker;
   R₁ is a group that is reactive with sulfenylated proteins;
   L₂ is a second linker;
   L₃ is a branched linker; and
   R₂ is a cellular directing group (e.g., fructose, folic acid, or diethyl amino coumarin),
   or a pharmaceutically acceptable salt thereof.

Further provided is a compound as taught herein for use in labeling tissues in a subject (e.g., PET imaging of a subject), for treatment of cancer, and/or for preparing a medicament.

### BRIEF DESCRIPTION OF THE DRAWINGS

FIG. 1: Primary characterization of the SCC-61/rSCC-61 system with respect to: (A) response to radiation, (B) response to EGFR inhibitors, (C) cytosolic and mitochondrial H₂O₂ and protein biomarker of DNA damage, and (D) expression of antioxidant proteins.

FIG. 2: The oxidation probe BP1 shows lower protein oxidation content in clinical biopsies from head and neck cancer (HNC) patients resistant to radiation treatment (***, p<0.001).

FIG. 3: (A) Synthesis of first protein oxidation tracer [¹⁸F]-DCP. (B) HPLC analysis of [¹⁸F]-DCP (upper chromatogram) and F-DCP control (lower chromatogram). (C) Ex vivo serum stability assay for [¹⁸F]-DCP. (D) [¹⁸F]-DCP uptake in SCC-61 and rSCC-61 cells. (E) Binding specificity using blocking with non-radioactive analog.

FIG. 4: In vivo biodistribution and mPET studies with the [¹⁸F]DCP prototype. (A) Tumor uptake. (B) Tumor to muscle (T:M) ratio. (C) mPET imaging (IVIS luciferase imaging is shown in the inset).

FIG. 5: Measurements of glucose uptake (A), mitochondrial membrane potential (B) redox parameters ratios (C) and vascular hypoxia (D) using optical spectroscopy.

FIG. 6: (A) BP1 inhibits the growth of rSCC-61 tumors but not SCC-61 (25 mg/kg every 3 days). (B) BP1 does not impact weight over this period of time.

FIG. 7: (A) Time course of acute inflammation in infection. (B) Protein oxidation in heart transitions from an increase during anabolism (early sepsis, 6 h post-CLP) to a decrease during late sepsis (24 h post-CLP).

FIG. 8: Chemical structure of DCP-NEt₂C (A), kinetics of uptake in live cells, signal co-localization with mitochondria (C), and differential labeling of SCC-61 and rSCC-61 cells under basal and increased oxidative stress induced by tBHP (tertbutyl hydroperoxide) and MitoPQ (D).

FIG. 9: Gene expression analysis of FOLR1 and GLUT5 (UGCG) in radiation resistant versus sensitive tumors extracted from TCGA for HNC (A) and across cancers (B).

### DETAILED DESCRIPTION

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

The disclosures of all patent references cited herein are hereby incorporated by reference to the extent they are consistent with the disclosure set forth herein. As used herein in the description of the invention and the appended claims, the singular forms “a,” “an” and “the” are intended to include the plural forms as well, unless the context clearly indicates otherwise.

“Label” as used herein may be any suitable label or detectable or otherwise functional group, including but not limited to biotin, avidin, fluorophores, antigens (including proteins and peptides), antibodies, porphyrins, radioactive or stable isotopes, etc.

“Linker” or “linking group” as used herein may be any suitable linking group, including but not limited to groups comprising, consisting of or consisting essentially of C, 0, N, P and/or S (e.g., including H where necessary). Linking groups that may be used to form covalent conjugates of two functional moieties are known in the art. See, e.g., U.S. Pat. Nos. 6,420,377; 6,593,334; and 6,624,317. The specific linking group employed will depend upon the particular synthetic method used to make the covalent conjugate, as will be appreciated by those skilled in the art. A suitable linking group will permit the joining of groups to provide a metabolically stable conjugate. In general, the linking moiety may comprise an aliphatic, aromatic, or mixed aliphatic and aromatic group (e.g., alkyl, aryl, alkylaryl, etc.) and contain one or more amino acids or hetero atoms such as N, O, S, etc.

Subjects, tissues, cells, cell fractions, and proteins utilized to carry out the present invention may be of any suitable source including microbial (including gram negative and gram positive bacteria, yeast, algae, fungi, protozoa, and viral, etc.), plant (including both monocots and dicots) and animal (including mammalian, avian, reptile, and amphibian species, etc.).

Mammalian subjects include both humans and other animal species treated for veterinary purposes (including but not limited to monkeys, dogs, cats, cattle, horses, sheep, rats, mice, rabbits, goats, etc.).

### I. Active Compounds Useful for PET Imaging.

The present invention provides compounds of Formula I:

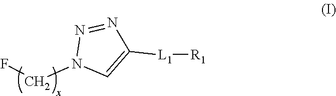

wherein:
   F is fluoro;
   x is an integer of from 1 to 10;
   L₁ is a linker; and
   R₁ is a group that is reactive with sulfenylated proteins,
   or a pharmaceutically acceptable salt thereof.

Also provided is a compound of Formula II:

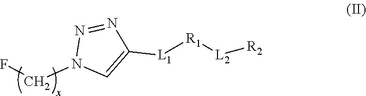

wherein:
   F is fluoro;
   x is an integer of from 1 to 10;
   L₁ is a first linker;
   R₁ is a group that is reactive with sulfenylated proteins;
   L₂ is a second linker; and
   R₂ is a cellular directing group (e.g., fructose, folic acid, or diethyl amino coumarin), or a pharmaceutically acceptable salt thereof.

Further provided is a compound of Formula III:

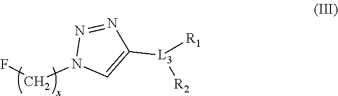

wherein:
   F is fluoro;
   x is an integer of from 1 to 10;
   L₃ is a branched linker;
   R₁ is a group that is reactive with sulfenylated proteins; and
   R₂ is a cellular directing group (e.g., fructose, folic acid, or diethyl amino coumarin), or a pharmaceutically acceptable salt thereof.

In some embodiments of the compounds, the fluoro is fluorine-18.

It will be appreciated that other PET compatible radioisotopes may be used in the molecule instead of fluorine-18 in accordance with the present invention. Such other PET compatible radioisotopes include, but are not limited to, carbon-11, gallium-68, zirconium-89, copper-64, etc. As non-limiting examples, carbon-11 or gallium-68 may be attached at the end of the molecule similar to fluorine-18. Copper-64 or zirconium-89 may be attached at the aromatic closed ring system.

In general, the linking moiety (L₁ and L₂ or L₃ when present) may comprise an aliphatic, aromatic, or mixed aliphatic and aromatic group (e.g., alkyl, aryl, alkylaryl, etc.) and contain one or more amino acids or hetero atoms such as N, O, S, etc. In some embodiments, L₁ and/or L₂ is a C1-C10 alkyl, a ketone, an amide, an ester, a carbamate, a urea, an ether (e.g., polyethylene glycol), a carbonate, or a combination of two or more thereof. For example, the linking group L₁ and/or L₂ may be a compound of the formula La-Lb, where Lb is present or absent and La and Lb are each independently selected from the group consisting of: a C1-C10 alkyl, a ketone, an amide, an ester, a carbamate, a urea, an ether, a carbonate, etc.

For example, L₁ may have the formula L₁ₐ-L_{1b}-L_{1c}, wherein L₁ₐ is selected from the group consisting of —(CH₂)ₙ—,

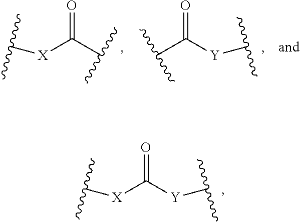

wherein n is an integer of from 1 to 6 and wherein X and Y are each independently selected from C, O, N and S (e.g., forming a ketone, carbamate, amide, urea, or carbonate group); and L_{1b} and L_{1c} are each independently present or absent and when present is selected from the group consisting of —(CH₂)ₙ—,

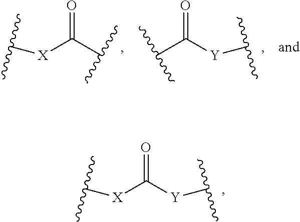

wherein n is an integer of from 1 to 6 and wherein X and Y are each independently selected from C, O, N and S (e.g., forming a ketone, carbamate, amide, urea, or carbonate group).

For example, L₂ may have the formula L₂ₐ-L_{2b}-L_{2c}, wherein L₂ₐ is selected from the group consisting of —(CH₂)ₙ—,

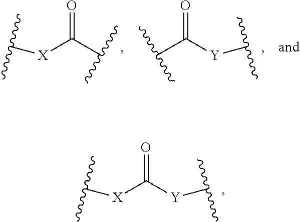

wherein n is an integer of from 1 to 6 and wherein X and Y are each independently selected from C, O, N and S (e.g., forming a ketone, carbamate, amide, urea, or carbonate group); and L_{2b} and L_{2c} are each independently present or absent and when present is selected from the group consisting of —(CH₂)ₙ—,

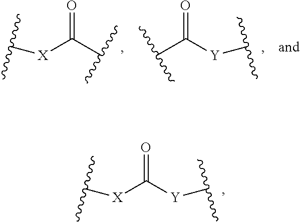

wherein n is an integer of from 1 to 6 and wherein X and Y are each independently selected from C, O, N and S (e.g., forming a ketone, carbamate, amide, urea, or carbonate group).

For example, L₃ may be a branched linking group such as a branched C1-C10 alkyl, an aryl group, or a heteroaryl group, optionally substituted with a linking moiety provided above for L₁ or L₂.

Further examples of suitable linking groups may be found in U.S. Pat. No. 7,294,749 to Poole et al., the contents of which is incorporated by reference herein.

In some embodiments, R₁ comprises a 1,3 dicarbonyl group or a bicyclononyne.

In some embodiments, R₁ is selected from the group consisting of:

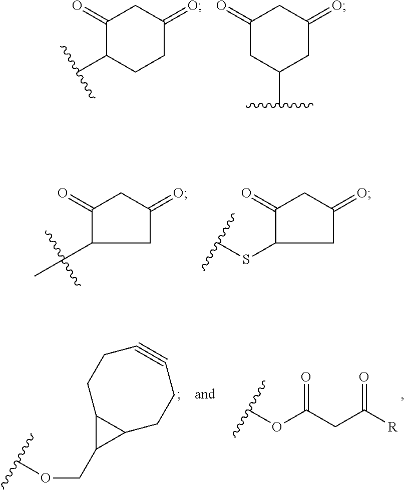

wherein R is an alkyl, ester or amide group. See also U.S. Pat. No. 9,023,653 to Furdui et al., which is incorporated by reference herein.

In some embodiments, R₁ is selected from the group consisting of:

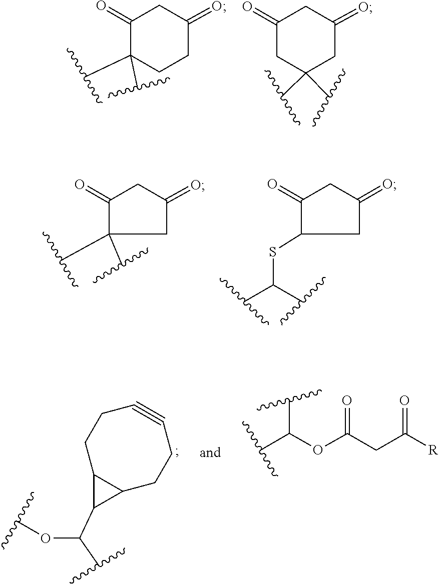

wherein R is an alkyl, ester or amide group.

In some embodiments, R₂ is selected from the group consisting of:

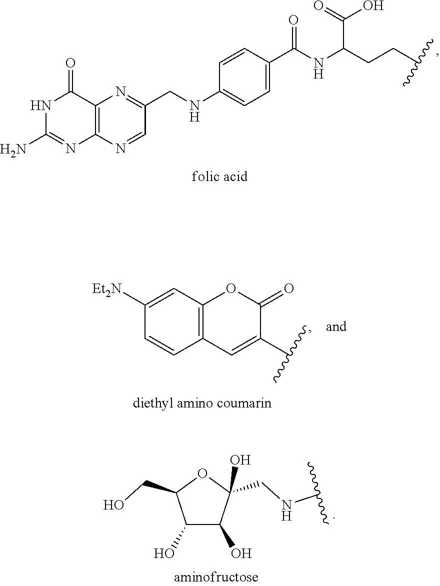

A particular example of a compound of Formula III is:

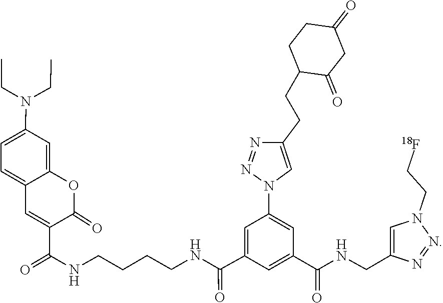 [SMILES: CCN(CC)c1ccc2cc(C(=O)NCCCCNC(=O)c3cc(C(=O)NCc4cn(CC[18F])nn4)cc(-n4cc(CCC5CCC(=O)CC5=O)nn4)c3)c(=O)oc2c1]

Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (b) salts formed from elemental anions such as chlorine, bromine, and iodine.

The compounds may be included in formulations suitable for oral, rectal, topical, buccal (e.g., sub-lingual), parenteral (e.g., subcutaneous, intramuscular, intradermal, or intravenous) and transdermal administration. Formulations of the present invention suitable for parenteral administration may conveniently comprise sterile aqueous preparations of the active compound, which preparations are preferably isotonic with the blood of the intended recipient. These preparations may be administered by means of subcutaneous, intravenous, intramuscular, or intradermal injection. Such preparations may conveniently be prepared, for example, by admixing the compound with water or a glycine buffer and rendering the resulting solution sterile and isotonic with the blood.

### II. Methods of Synthesis and Precursor Compounds.

Methods of synthesizing a compound as taught herein may include one or more of the steps of:
reacting a compound of formula A: N₃—(CH₂)ₓ₊₁—Z, wherein Z is a leaving group (e.g., tosylate or mesylate), and x is as defined above, with fluorine (e.g., F-18) or other PET imaging compatible radioisotope, or the respective non-radioactive atom, to form a compound: N₃—(CH₂)ₓ₊₁—F;
providing an alkyne-containing compound of formula B:

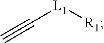

and then
reacting the compound N₃—(CH₂)ₓ₊₁—F with the alkyne-containing compound of formula B, said reacting carried out by a copper-catalyzed Click reaction.

In some embodiment, the fluoro is fluorine-18.

As noted above, it will be appreciated that other PET compatible radioisotopes may be used instead of fluorine-18 in accordance with the present invention. Such other PET compatible radioisotopes include, but are not limited to, carbon-11, gallium-68, zirconium-89, copper-64, etc.

Example synthetic methods for some embodiments of the active compounds are as follows:

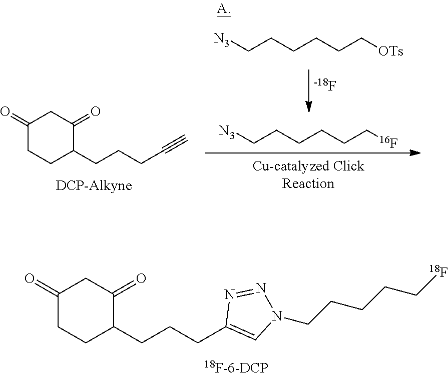

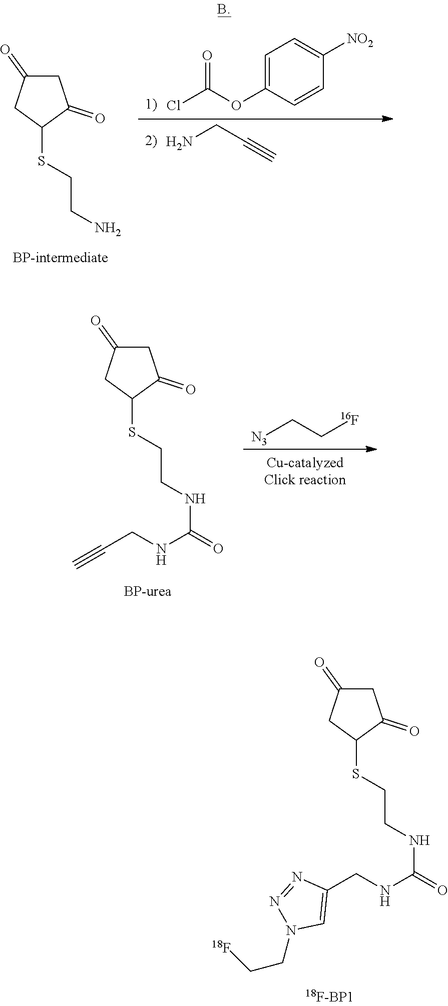

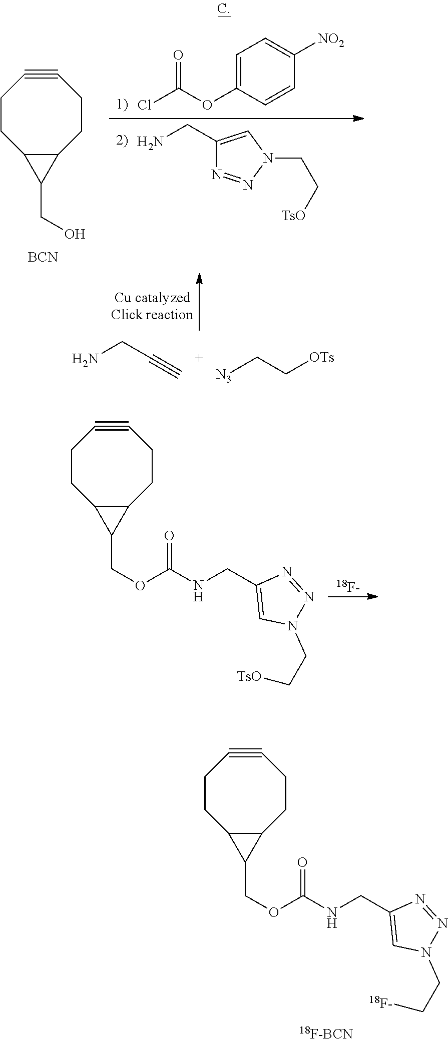

### Schemes A, B and C.

Shown in the schemes above is the synthesis of [¹⁸F]6DCP, a more hydrophobic analog. Also depicted is the synthesis of [¹⁸F]BP1 and [¹⁸F]BCN, respectively. Conversion of a known BP-synthetic intermediate to an alkyne-containing BP-urea followed by the Click reaction of N3CH2CH2[¹⁸F] will yield [¹⁸F]BP1. The presence of a strained alkyne in BCN interferes with [¹⁸F] introduction by Click chemistry. The proposed route to [¹⁸F]BCN pre-clicks propargyl amine with an azide-containing tosylate (OTs) to give an amino triazole and condensation with activated BCN yields a carbamate. Fluoride ion displacement of the tosylate will give [¹⁸F]BCN. These sequences yield triazole products that possess UV absorbance assisting in their identification and HPLC purification. Non-radioactive versions of each of these compounds may also be prepared using these strategies, and may be characterized, e.g., by NMR spectroscopy (hydrogen, carbon and fluorine), MS etc. These standards can aid in the identification and purification of the labeled species by HPLC.

In some instances, the 1,3-dicarbonyl group may also act as a ligand for the copper catalyst. One of skill in the art will appreciate that the order of reaction can be switched with the Click reaction being performed initially (e.g., using the tosylate) followed by displacement with [¹⁸F].

Further example synthetic methods for some embodiments of the active compounds are as follows:

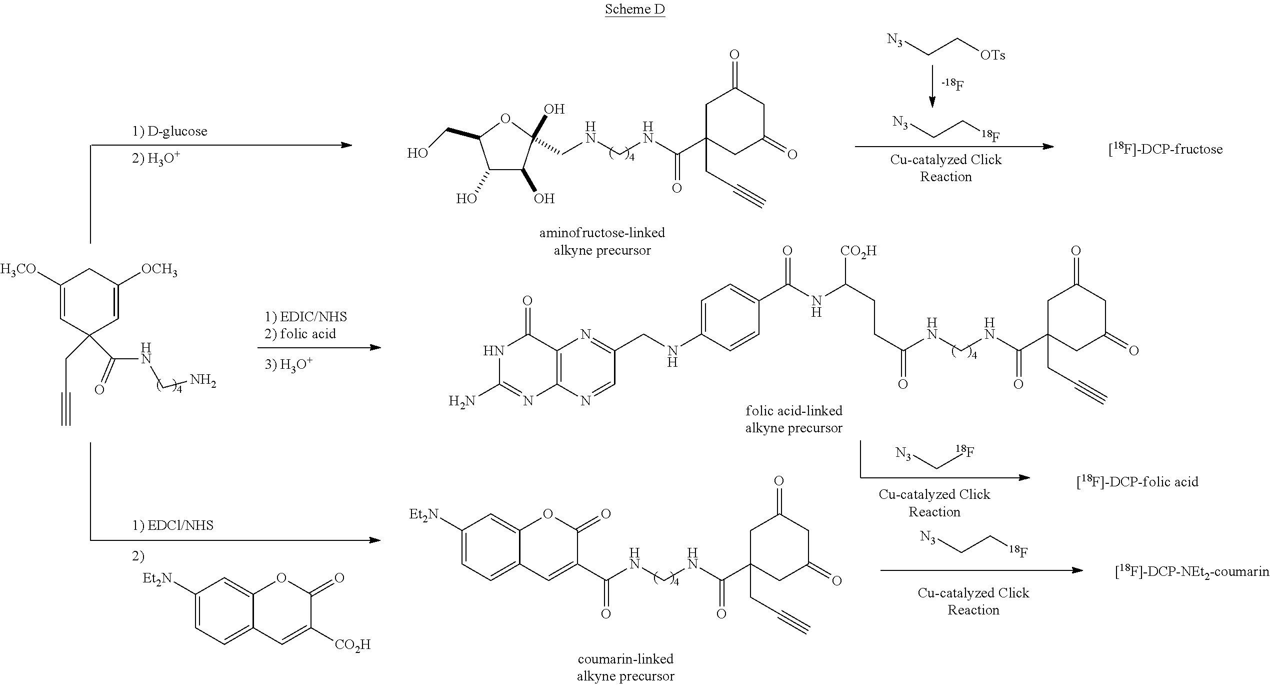

Shown in Scheme D above is the synthesis of [¹⁸F]DCP-fructose, [¹⁸F]DCP-folic acid and [¹⁸F]DCPNEt₂-coumarin.

Additional example synthetic methods for some embodiments of the active compounds are as follows:

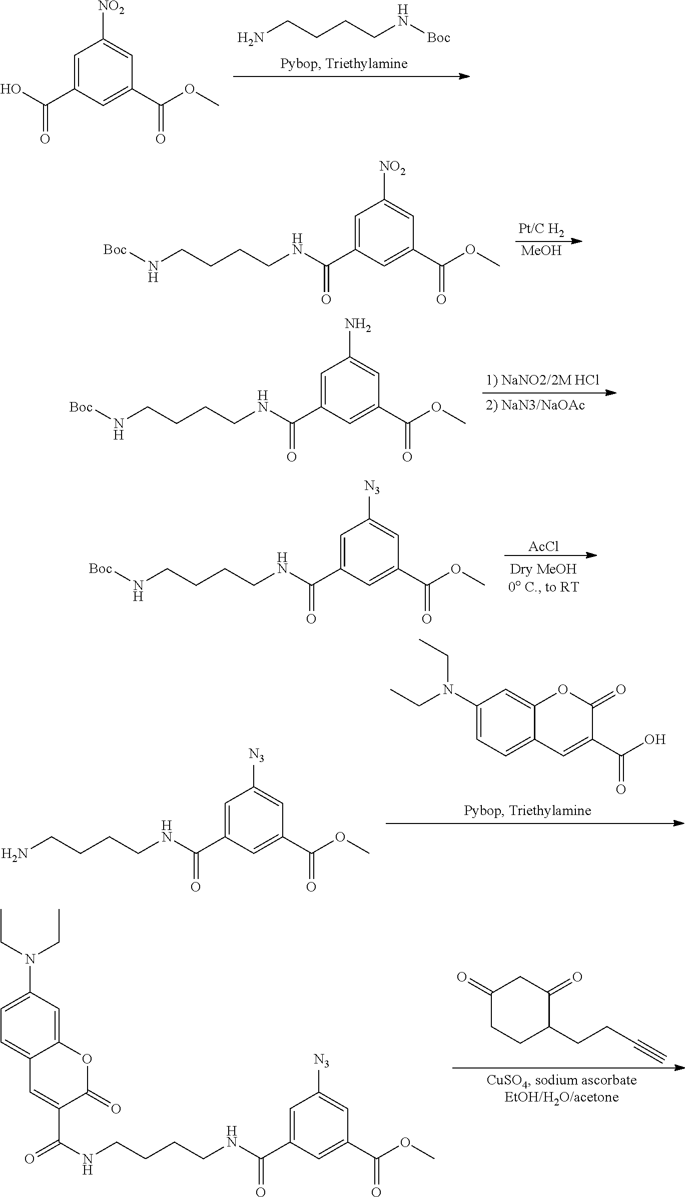

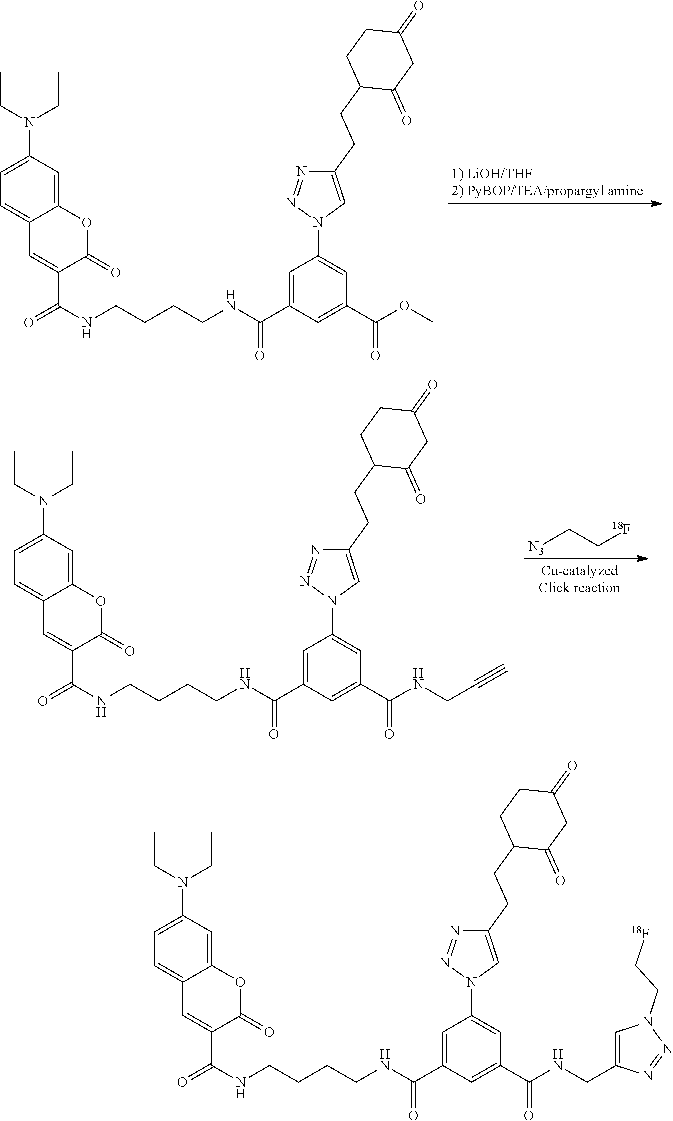 [SMILES: CCN(CC)c1ccc2cc(C(=O)NCCCCNC(=O)c3cc(C(=O)OC)cc(-n4cc(CCC5CCC(=O)CC5=O)nn4)c3)c(=O)oc2c1 ; C#CCNC(=O)c1cc(C(=O)NCCCCNC(=O)c2cc3ccc(N(CC)CC)cc3oc2=O)cc(-n2cc(CCC3CCC(=O)CC3=O)nn2)c1 ; [N-]=[N+]=NCC[18F] ; CCN(CC)c1ccc2cc(C(=O)NCCCCNC(=O)c3cc(C(=O)NCc4cn(CC[18F])nn4)cc(-n4cc(CCC5CCC(=O)CC5=O)nn4)c3)c(=O)oc2c1]

Also provided are precursor compounds useful to synthesize the compounds as taught herein, for example, precursor compound D:

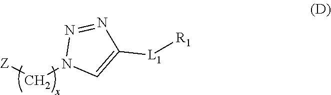

wherein:
   Z is a leaving group;
   x is an integer of from 1 to 10;
   L₁ is a linker; and
   R₁ is a group that is reactive with sulfenylated proteins.

Also provided are precursor compounds E and F:

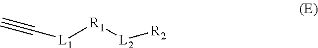

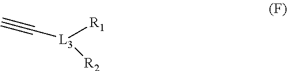

wherein:
   L₁ is a first linker;
   R₁ is a group that is reactive with sulfenylated proteins;
   L₂ is a second linker;
   L₃ is a branched linker; and
   R₂ is a cellular directing group (e.g., fructose, folic acid, or diethyl amino coumarin), or a pharmaceutically acceptable salt thereof.

Particular examples of precursor compound E include, but are not limited to:

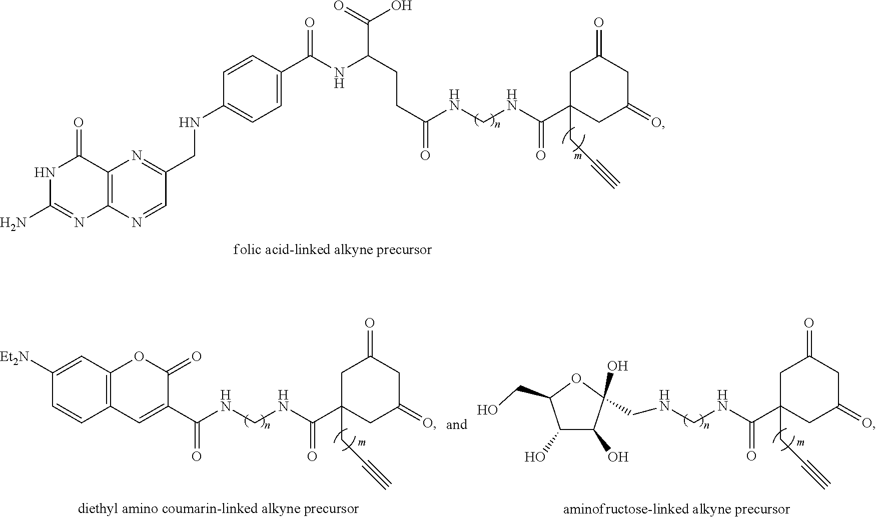

wherein n and m are each independently an integer of from 1 to 10.

### III. Methods of Use for Imaging.

The active compounds taught herein are useful in methods of labeling tissues in a subject, particularly tissues with sulfenylated proteins, such as proteins carrying sulfenic acid (—SOH), sulfenylamides (—SN), or hypohalous and hypothiocyanous acids halogens (e.g., —Cl in —SOClX, —SOBr and —SOSCN) at selected (e.g., one or more) cysteine residues.

Sulfenylated proteins include proteins carrying sulfenic acid (—SOH), sulfenylamides (—SN), or hypohalous and hypothiocyanous acids (e.g., —SOCl, —SOBr and —SOSCN) at one or more of their cysteine residues. See, e.g., Qian et al., “A simple and effective strategy for labeling cysteine sulfenic acid in proteins by utilization of beta-ketoesters as cleavable probes,” Chem. Commun. 2012; 47(32):4091-3; Gupta et al., “Profiling the reactivity of cyclic C-nucleophiles towards electrophilic sulfur in cysteine sulfenic acid,” Chem. Sci. 2106; 7:400-415.

Such methods of labeling may include steps of administering an active compound as taught herein to a subject, and then performing a PET scan on the subject, wherein the PET scan detects the presence or absence of binding of said compound to the tissues, the presence of binding indicating the presence of sulfenylated proteins in said tissues.

In some embodiments, the tissues comprise cancerous tissues for cancer detection or treatment. Cancers may include, e.g., head and neck cancer, breast, colon, lung, prostate, brain or liver cancer. Other potential applications include, but are not limited to, neuroimaging (e.g., Alzheimer's), cardiology (e.g., hibernating myocardium, atherosclerosis, ischemia/reperfusion injury), infectious disease (e.g., infection-induced inflammatory response), and measuring musculoskeletal activity.

In some embodiments, the administering is carried out by parenteral administration.

For cancerous tissues in particular, the methods may further be used to determine whether a cancerous tissue has an increased likelihood of responding to radiation treatment, the presence of binding of the active compound indicating an increased likelihood of response of said cancerous tissue to radiation treatment.

Furthermore, the methods may be used to determine whether a cancerous tissue has an increased likelihood of responding to therapies targeting receptor tyrosine kinases, the presence of binding of the active compound indicating a decreased likelihood of response of said cancerous tissue to drug treatment. Non-limiting examples of such therapies include, but are not limited to, erlotinib, imatinib, afatinib, etc.

### IV. Administration of Non-Labeled Compounds for Treatment of Cancer.

In some embodiments, a compound containing a group that is reactive with sulfenylated proteins may be administered to a subject in need of treatment for cancer. For example, the subject may be administered a treatment-effective amount of a compound: L₁-R₁, or L₁-R₁-L₂-R₂,
wherein:
L₁ and L₂ are each independently present or absent, and when present is independently a C1-C10 alkyl, a ketone, an amide, an ester, a carbamate, a urea, an ether (e.g., polyethylene glycol), a carbonate, or a combination of two or more thereof, as described above;
R₁ is a group that is reactive with sulfenylated proteins; and
R₂ is a cellular directing group (e.g., fructose, folic acid, or diethyl amino coumarin),
or a pharmaceutically acceptable salt thereof.

Cancers may include, e.g., head and neck cancer, breast, colon, lung, prostate, brain or liver cancer.

Treatment may include administration of radiation therapy to the subject.

In some embodiments, the cancer is determined to be resistant to radiation treatment or treatment targeting receptor tyrosine kinases (e.g., by PET imaging with a ¹⁸F or other PET compatible radioisotope containing compound as taught herein).

For such purposes the compounds may be provided as a pharmaceutical formulation in a suitable pharmaceutical carrier, for example, an aqueous carrier such as sterile pyrogen-free water or saline solution. The pharmaceutical formulation may be administered to a subject (e.g., a human subject, or other mammalian subject such as a dog, cat, or monkey for veterinary purposes) afflicted with a cancer as noted above by any suitable means, typically parenterally (e.g., intravenous, subcutaneous, intraperitoneal injection, etc.) in a suitable amount (e.g., from 1, 2, 5 or 10 to 20, 30, 40 or 50 mg per kg; or from 0.08, 0.16, 0.4, or 0.81 to 1.6, 2.4, 3.2 or 4 mg/kg), alone or in combination with radiation therapy.

In some embodiments, the cancer is determined to be resistant to treatment with an epidermal growth factor receptor (EGFR) antagonist (e.g., erlotinib, imatinib, afatinib, etc.).

The present invention is explained in greater detail in the following non-limiting Examples.

### Examples

Redox metabolism is increasingly acknowledged as a critical factor in cancer development, resistance to treatment, metastasis and overall disease prognosis. Distinct tumor redox profiles are found along the time course of cancer management as detailed in one of our recent reviews. While a number of radiotracers for monitoring aspects related to reactive oxygen species (ROS) have been developed, these do not meet the requirements for clinical translation. Importantly, there are no published PET radiotracers or other imaging probes for tracking protein oxidation in vivo.

Our objective is to target tumor redox state for development of non-invasive imaging tools for clinical monitoring of tumor redox state by focusing on protein oxidation. We have previously demonstrated the application of 1,3-dicarbonyl (e.g., DCP, BP1) and bicyclononyne (BCN) reagents for quantification of protein oxidation (sulfenic —SOH modification) in cells and clinical specimens.

In our initial research, we focused on Head and Neck Cell Cancer (HNC), a highly heterogeneous disease occurring at multiple locations over the upper aerodigestive tract and for which there is a lack of unified imaging methods for early detection and prediction of response to treatment. HNC is a largely preventable disease with major etiologic factors being smoking, chewing tobacco, excessive alcohol consumption, and infection with Human Papillomavirus (HPV). These agents induce ROS as a major mechanism of their carcinogenic potential. Studies have shown clear benefits of early detection in improving overall survival and patients' quality of life as treatment of smaller tumors would have a better chance of sparing critical anatomical parts such as salivary glands and vocal cords. Unfortunately, while some tools exist for early detection of cancer, they have significant limitations and vary widely depending on the tumor site. No tools exist that can be applied uniformly for imaging of the entire upper aerodigestive system. Second, radiation therapy is often used in treatment of HNC patients typically in combination with surgery and/or chemotherapy. Evaluation for treatment response is generally performed 12 weeks after the end of the definitive treatment intervention with PET or CT scan with contrast. Long-term follow up is by regular direct clinical evaluations every 2-3 months during the first year, every 3-4 months during the second year and every 6 months in the next 3 years. The tools used for detection depend on the site that is being monitored sometimes complemented by [¹⁸F]FDG PET/CT as needed. While changes in [¹⁸F]FDG before and after radiation treatment can be predictive of risk of recurrence, this approach still requires patients to undergo radiation treatment. Currently, there is a lack of imaging methods to predict the patients most likely to benefit from radiation before the start of treatment.

Reactive oxygen species (ROS), including hydrogen peroxide (H₂O₂) are tightly associated with all aspects of cancer. ROS are known to mediate cancer development: environmental pollutants, infection with viruses or pathogenic bacteria, chronic inflammation and diet/microbiota generate ROS, and are well-established factors of malignant transformation. ROS further support tumor growth by facilitating angiogenesis and cell invasion processes. ROS and their metabolism have also been linked to mitochondrial activity facilitating intra- and inter-cellular communication within the tumor cells or tumor microenvironment, and long range communication during metastasis. Effective treatment of cancer with radiation and chemotherapies typically relies on accumulation of ROS, which induce oxidative stress and cell death. However, resistance to treatment is often associated with upregulation of the antioxidant systems and a more reduced state.

ROS exert their effects through targeted oxidation of cellular macromolecules including proteins controlling signaling, metabolism and epigenetics. The oxidation of these proteins occurs with high selectivity at either catalytic or regulatory cysteine sites. The scientific premise of our approach is that imaging of selective protein oxidative modifications imparted by ROS last longer and carry more molecular significance than detection of ROS levels alone. The imaging tools proposed here are selective for a specific oxidative modification, sulfenylation at unique reactive cysteine sites in proteins.

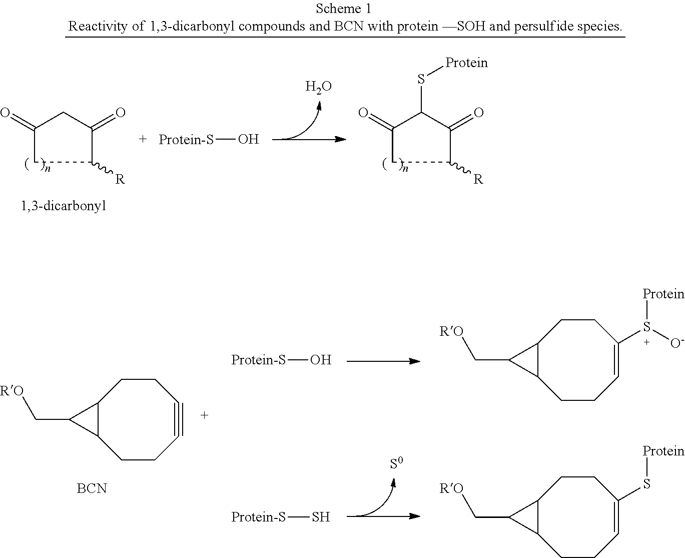

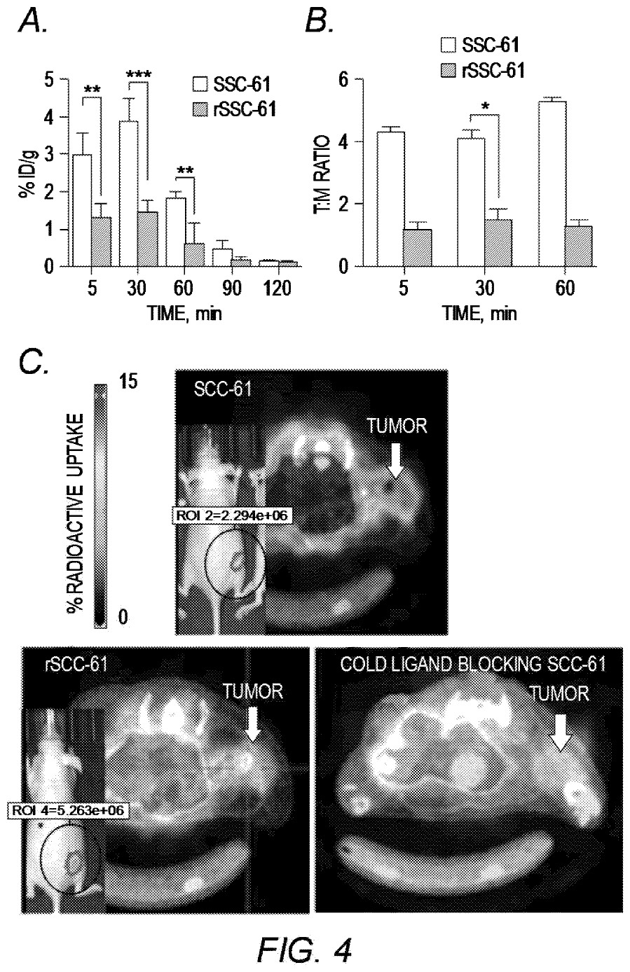

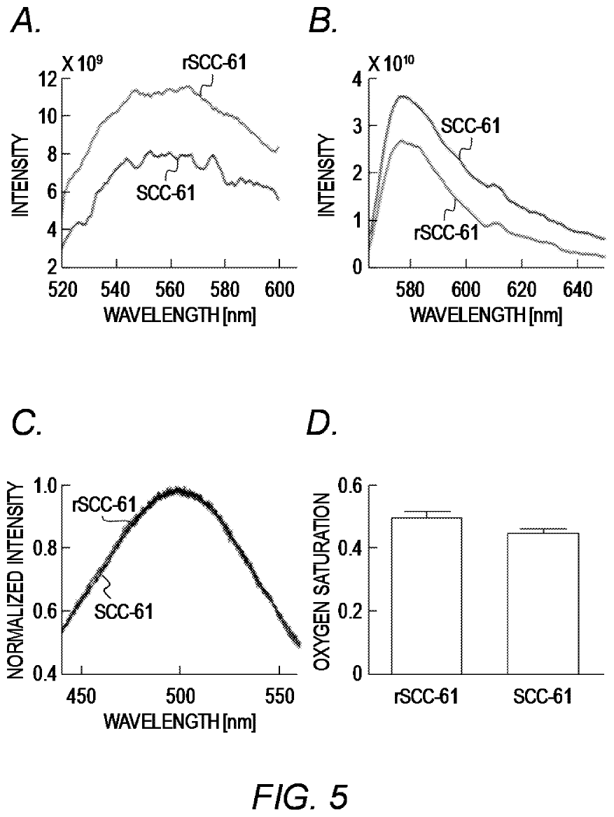

Detection of radiation sensitivity. None of the current radiotracers monitor protein oxidation, and target, instead, surrogate ROS levels, availability of reducing equivalents, glucose uptake, and tumor oxygenation. However, these have significant limitations. [¹⁸F]FDG is not predictive of radiation response, and hypoxia tracers are not suitable for early detection. While change in [¹⁸F]FDG collected before and after radiation treatment can be used to predict risk of recurrence, this approach still requires patients to undergo treatment with radiation. By contrast, our approach may identify radiation resistant tumors before radiation treatment is attempted. Also, while highly hypoxic tumors tend to be more resistant to radiation and most other therapies, radiation sensitive tumors also have hypoxic regions. Most importantly, the relationship between pO2 and ROS is quite complex and low pO2 does not necessarily mean low protein sulfenylation. Our data show a large difference in protein sulfenylation between tumors with similar degrees of oxygenation (FIG. 4 and FIG. 5).

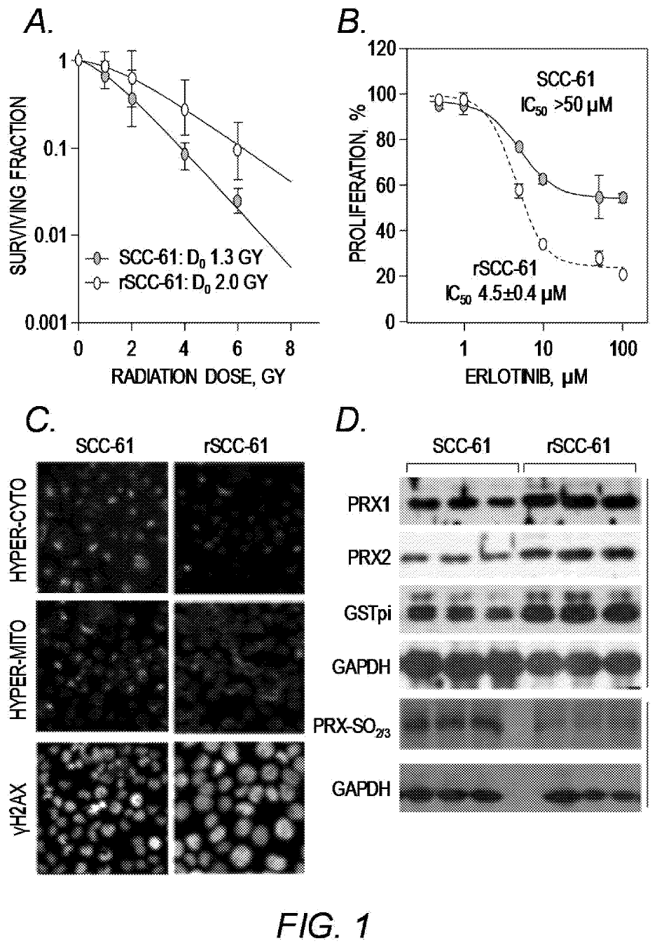

Initial characterization of the SCC-61/rSCC-61 cells (genetically matched model of radiation resistance in HNC) showed differences in: 1) response to radiation (SCC-61: DO 1.3, rSCC-61 DO 2.0) (FIG. 1A); 2) response to the EGFR targeted inhibitor Erlotinib (SCC-61: IC50 >50 μM; rSCC-61: IC50 4.5 μM) (FIG. 1B); and, 3) cellular phenotype; SCC-61: mesenchymal; rSCC-61: epithelial (not shown). See also Bansal et al., “Broad Phenotypic Changes Associated with Gain of Radiation Resistance in Head and Neck Squamous Cell Cancer,” Antioxidants & Redox Signaling vol. 21(2):221-236. Quantitative proteomics revealed: 1) upregulation of antioxidant proteins (Prx1-3, GST-pi) and downregulation of MnSOD in rSCC-61; cumulatively these results explain the differences in intracellular ROS in rSCC-61 (low) and SCC-61 (high) (FIG. 1C; and FIG. 1D shows validation of proteomics data by western blot); 2) upregulation of DNA Replication and Base Excision Repair in rSCC-61 correlating with less DNA damage in rSCC-61; 3) downregulation of ECM-Receptor Interaction and Focal Adhesion in rSCC-61; and, 4) upregulation of keratins (relevant for epithelial phenotype), fatty acid synthase (relevant for lipid and bioenergy metabolism) and C1 metabolism (relevant for NADPH/NADP+ balance, SAM biosynthesis and DNA methylation).

Using redox proteomics and related studies we have found decreased oxidation (therefore increased activation) of antioxidant proteins like peroxiredoxins in rSCC-61, which translated into decreased overall protein oxidation in rSCC-61 cells (not shown). Considering the upregulation of metabolic pathways in rSCC-61, we followed up with quantitative metabolomics, lipidomics and bioenergy analysis. Bioenergy analysis showed funneling of glucose metabolism into the Pentose Phosphate Pathway in rSCC-61 consistent with the increased NADPH/NADP+ ratios (SCC-61: 11.1; rSCC-61: 17.5), oxygen consumption rates (OCR) (SCC-61: 169 pmoles/min; rSCC-61: 96 pmoles/min) and ATP synthesis (SCC-61: 155 pmoles/min; rSCC-61: 83 pmoles/min) in these cells. Computational modeling of the multi-omics data and data collected from the TCGA and Human Protein Atlas for HNC pointed to a mechanistic model of radiation resistance where ROS metabolism and protein oxidation are at the core of resistance to radiation and are controlled by lipid metabolism and the flux of NADP+ and NADPH metabolites. Taken together, the findings further support the prediction of response to radiation by probing for protein oxidation.

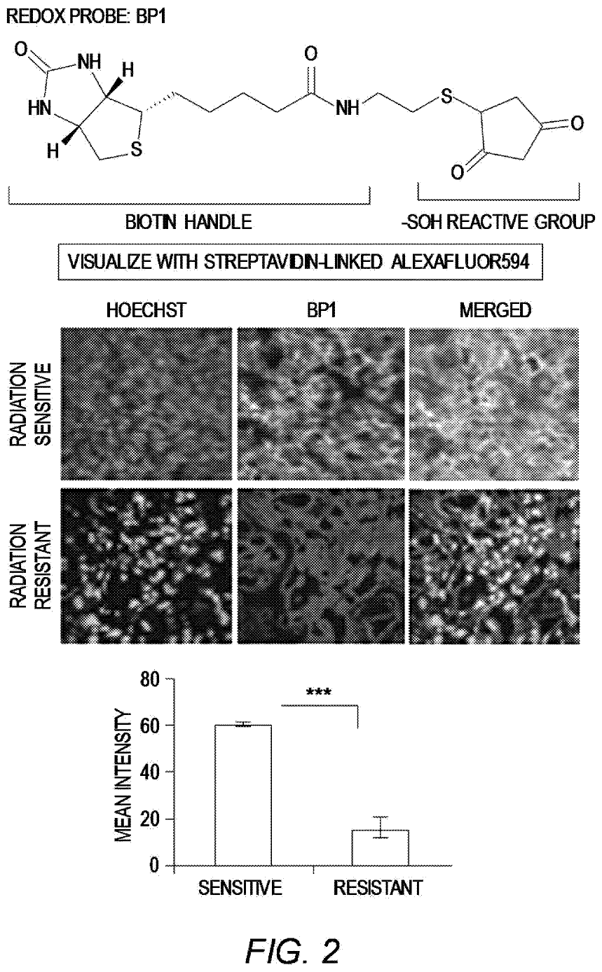

To further investigate this, we performed retrospective analysis of protein oxidation in HNC clinical samples using the protein oxidation sensing probe BP1 (a 1,3-dicarbonyl probe). As shown in FIG. 2, HNC tumors from patients who responded to radiation treatment had increased staining with BP1 compared with resistant tumors consistent with ROS and redox proteomics analysis of SCC-61/rSCC-61 cells. This demonstrates the significance of following protein oxidation with PET radiotracers as a potential biomarker of response to radiation or chemoradiation treatment.

### Synthesis of [¹⁸F]DCP and Characterization of Cellular Uptake and Serum Stability.

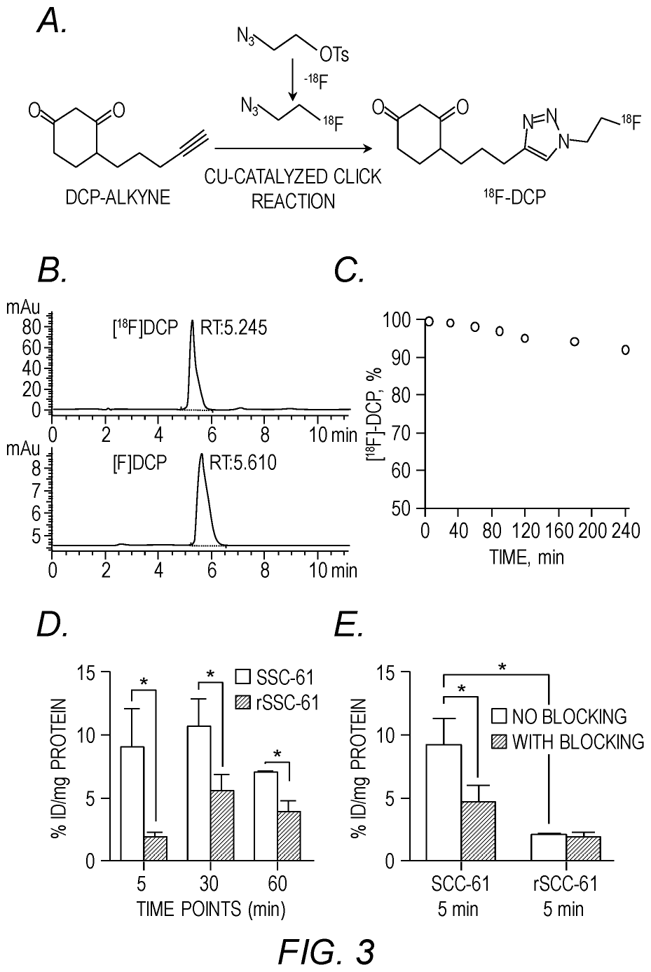

Based on our previous preparation of DCP-alkyne and the ability to directly use Click chemistry to add [¹⁸F] to molecules via N₃CH₂CH₂[¹⁸F], Click coupling of this known azide was performed with DCP-alkyne (FIG. 3A). The chemical and radiochemical purity of the [¹⁸F]DCP was verified by HPLC injection using the non-radioactive F-DCP standard as control (FIG. 3B). Further ex vivo serum stability of [¹⁸F]DCP was determined over a 6 h period (FIG. 3C). Uptake of [¹⁸F]DCP in SCC-61 cells (high intracellular ROS and protein oxidation) and rSCC-61 cells (low intracellular ROS and protein oxidation) was followed at 5, 30 and 60 min of radiotracer exposure. The results are shown in FIG. 3D and are expressed as % injected dose (ID)/mg of protein present in each well with p values≤0.05 (n=3). Receptor blocking experiments were also performed by exposing the cells to 50× excess non-radioactive F-DCP, 5.0 min prior to adding the [¹⁸F]DCP (FIG. 3E, p<0.05 for SCC-61, n=3).

In Vivo Evaluation of [¹⁸F]DCP. Female athymic nude mice were injected subcutaneously in the right flank with SCC-61 or rSCC-61 cells expressing a luciferase construct. The presence of viable tumors was confirmed through bioluminescence imaging. Biodistribution experiments were performed in SCC-61 and rSCC-61 tumor bearing mice. [¹⁸F]DCP (60-80 μCi, specific activity 2,610-2,820 mCi/μmol EOS) was administered through tail vein injection. Mice were sacrificed at 5, 30, 60, 90 and 120 min (n=4 for each time point) post radiotracer injection. The organs of interest including blood, brain, tumor, heart, lungs, liver, spleen, kidneys, pancreas, muscle and bone were removed, weighed, and counted in an automatic γ-counter. Radiotracer uptake in different organs was calculated as percentages of injected dose per gram of tissue (% ID/g tissue). The data showed that the radioligand had a differential uptake between SCC-61 and rSCC-61 tumors (FIG. 4A) i.e, >60% higher uptake in SCC-61 tumors over rSCC-61 tumors in mice. The results show excellent tumor to muscle ratio with >80% enrichment in the tumor. Additionally, the radioactivity peaked at 30 min in the target organ and gradually washed out from the blood pool by 120 min (kidney being the primary excretory organ had a minimal uptake) demonstrating favorable pharmacokinetics for tracer quantification. There was also no evidence of bone uptake suggesting lack of defluorination. mPET imaging of [¹⁸F]DCP in tumor bearing mice: mPET/CT imaging experiments were performed in both SCC-61 and rSCC-61 tumor bearing mice (n=3) using Trifoil PET/CT scanner (1.2 mm resolution and 14 cm axial field of view). [¹⁸F]DCP was intravenously injected and scanned 45 min post injection. A 3-min CT scan was obtained prior to the PET scan (20 min). Two SCC-61 tumor bearing mice from the same cohort were used for blocking experiments. These two mice received 10 mg/kg/i.v/100 μL of the unlabeled F-DCP, 20 min prior to the radiotracer injection. The images were reconstructed and analyzed using TriFoil attenuation correction and Fourier rebinning parameters. mPET results demonstrate excellent tumor uptake of [¹⁸F]DCP in SCC-61 tumor mice over the rSCC-61 ones, corroborating well with the biodistribution results. The tumor uptake was significantly blocked with the pretreatment of cold ligand F-DCP, evidencing the high specificity of the radioligand (FIG. 4C). In parallel studies, we have also monitored glucose uptake, mitochondrial membrane potential, redox state based on the FAD and NADH autofluorescence, and vascular parameters using optical spectroscopy in mice carrying subcutaneous SCC-61 and rSCC-61 tumors (FIG. 5). Consistent with our published cell culture data, the radiation resistant rSCC-61 tumors had increased glucose uptake (FIG. 5A) and decreased mitochondrial activity reflected in the membrane potential (FIG. 5B). Both tumors were hypoxic with similar vascular endpoints. Oxygen saturation is shown in FIG. 5D, and a similar profile was obtained for hemoglobin concentration (not shown). Interestingly, the autofluorescence measurement of FAD and NADH generated an identical profile for the SCC-61 and rSCC-61 tumors (FIG. 5C). This illustrates that unlike [¹⁸F]DCP, the [FAD]/([FAD]+[NADH]) redox ratio does not discriminate between these tumors further supporting the value added by our proposed strategy.

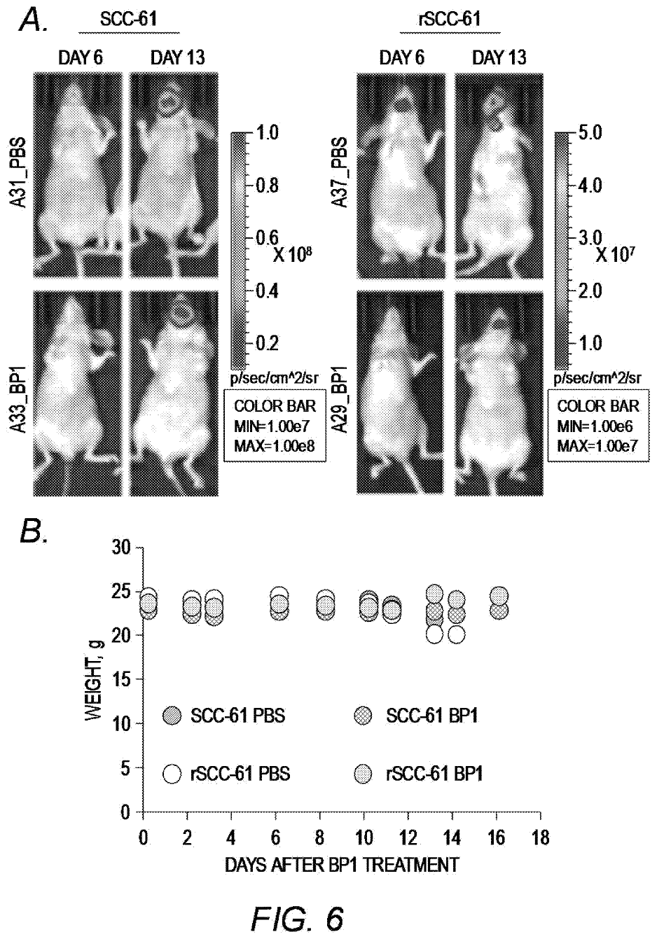

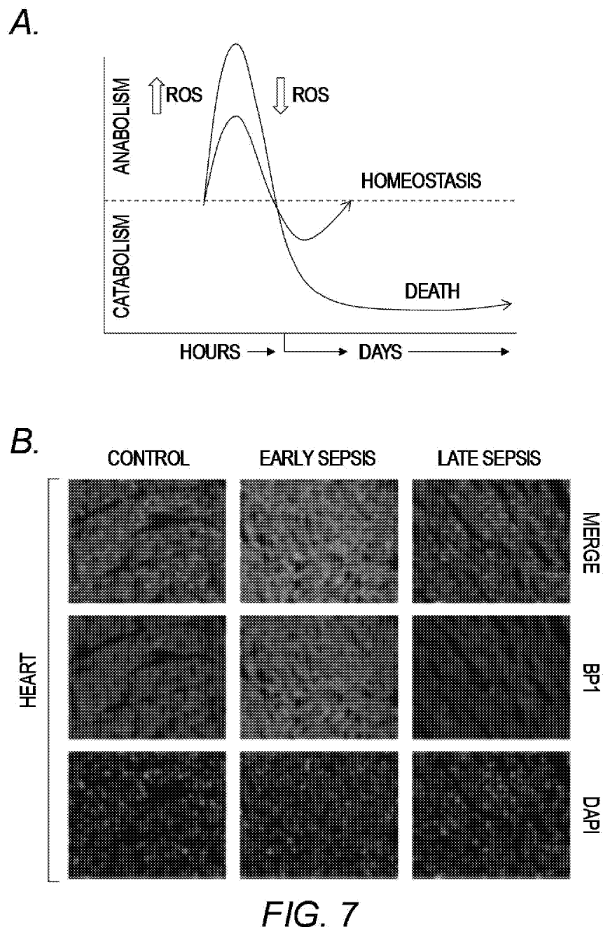

Complementary In Vivo Data Showing Lack of Toxicity and Sensitivity to Detect Inflammation. Mice carrying orthotopic SCC-61 and rSCC-61 tumors were injected either with BP1 25 mg/kg or vehicle after tumor establishment and the treatment was repeated every 3 days for 2-3 weeks. The results showed that rSCC-61 tumors had increased sensitivity to BP1 (FIG. 6A) and that BP1 treatment was not toxic to the animals as evidenced by lack of weight loss and adverse effects (FIG. 6B). As tumor initiation is often associated with inflammation, we sought to determine if it is possible to detect changes in inflammation associated with infection as proof of concept for the proposed application in early detection of cancer. We used a Cecal Ligation Puncture (CLP) and injected BP1 30 min prior to euthanasia. FIG. 7A shows theoretical redox profile with infection leading to sepsis and data in FIG. 7B shows BP1 labeling following this profile: increased protein sulfenylation during early/anabolic phase and decreased sulfenylation during late/catabolic in liver and heart (shown here).

Cell membrane permeability and toxicity. Preliminary cell culture and in vivo results shown here and our published data demonstrate cell membrane permeability and expected subcellular distribution of DCP compounds. Preliminary [¹⁸F]DCP data further support these conclusions. There has been no indication of toxicity for this class of compounds (FIG. 6B).

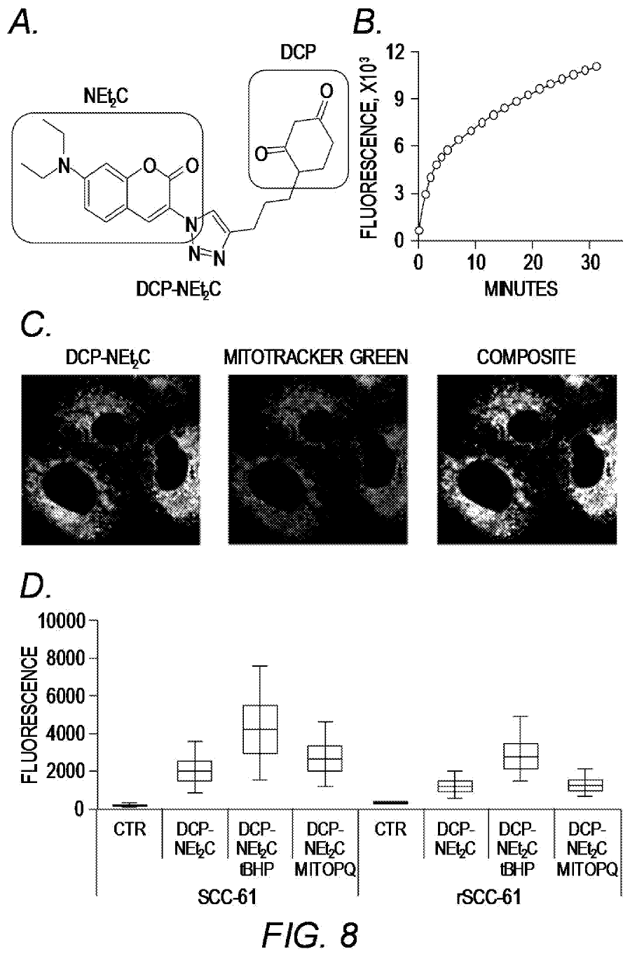

Targeting of Chemical Probes for Protein Oxidation to Mitochondria. We described in a recent publication a new fluorescent DCP reagent (DCP-NEt₂C), which is rapidly localized to mitochondria independent of mitochondria membrane potential and shows linear dependence of protein sulfenylation signal in both low and high ROS conditions. Following up on these studies, we applied DCP-NEt₂C to the SCC-61 and rSCC-61 cells and show again high capacity of this chemistry to discriminate between radiation sensitive and resistant cells (FIG. 8A-FIG. 8D).

Elaborated analogs of [¹⁸F]DCP targeted to cancer cells or mitochondria can be prepared containing [¹⁸F] and the non-radioactive control compounds. Scheme 2 shows the general synthetic strategy that requires assembly of a sulfenic acid reactive group with a chemical moiety targeting the radiotracer to cancer cells to further increase enrichment in tumors and discriminate between radiation resistant and sensitive tumors, or a mitochondria-directing group, along with a site for [¹⁸F] incorporation in the same molecule.

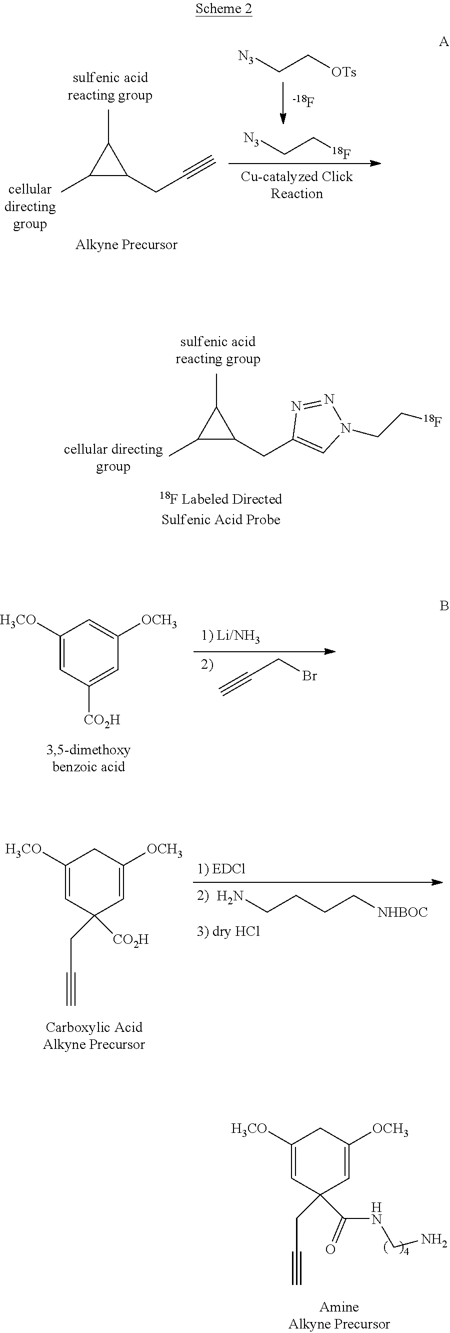

Similar to the synthesis of [¹⁸F]DCP, the Click reaction of N₃CH₂CH₂[¹⁸F](generated from N₃CH₂CH₂OTs) with the alkyne precursor introduces [¹⁸F] into the directed sulfenic acid probe (Scheme 2A). This approach allows for synthesis of a variety of analogs with varied properties in the linker region to increase or decrease hydrophobicity relative to [¹⁸F]DCP. Scheme 2B outlines the proposed synthesis of an amine alkyne precursor core required for the strategy presented in Scheme 2A. Birch reduction of commercially available 3,5-dimethoxy benzoic acid followed by alkylation with propargyl bromide will selectively give a 1, 4-cyclohexadiene carboxylic acid alkyne precursor (Scheme 2B). This sequence finds precedence in the preparation of a biotin-linked dimedone derivative for protein sulfenic acid detection. 1-Ethyl-3-(3-dimethylaminopropyl)carbodiimide (EDCI) promoted coupling of mono t-butoxycarbonyl (BOC)-protected 1,4-diaminobutane followed by BOC group removal with dry HCl gives an amine alkyne precursor (Scheme 2B). These alkynes contain a protected 1,3-dicarbonyl group as a double enol ether functionality while the carboxylic acid or amine groups allow the direct attachment of directing groups by standard peptide bond forming chemistry.

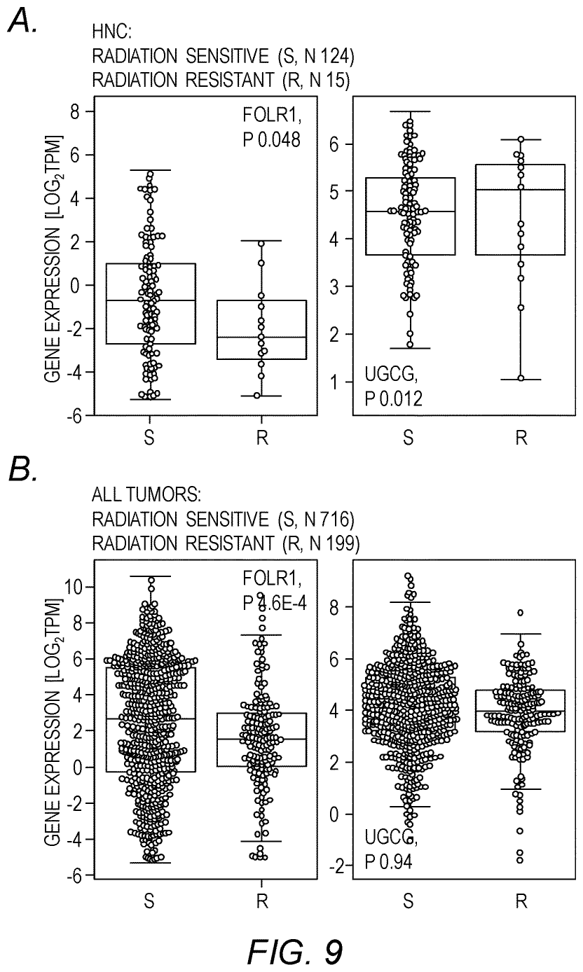

Further synthetic efforts will focus on producing PET probes that specifically identify sulfenylated proteins within cancer cells or mitochondria. Overexpression of fructose transport proteins (GLUT5) or folic acid receptors (e.g., FOLR1) in tumors provide a means for directing probes via attachment of fructose or folic acid. Analysis of FOLR1 and GLUT5 using TCGA data shows decreased expression of FOLR1 in radiation resistant cancers overall, including HNC (FIG. 9A). Thus, the lower uptake of a folate [¹⁸F]DCP derivative in resistant tumors coupled with decreased sulfenylation is expected to further broaden the PET difference between sensitive and resistant tumors. On the other hand, GLUT5 has increased expression in radiation resistant HNC with no difference across cancers (FIG. 9B). Thus, the folate and fructose-derivatives of [¹⁸F]DCP would provide value to understand the contribution of uptake to the PET signal and would take advantage of intrinsic differences in expression of endogenous receptors to improve prediction of radiation response.

Our recent work shows the diethyl amino coumarin moiety directs sulfenic acid traps to mitochondria. Scheme D in the detailed description above shows the proposed synthetic approach to cancer cell and mitochondria-directed sulfenic acid PET probes from a common amine alkyne precursor. Direct treatment of this amine alkyne precursor with glucose should yield an amino fructose linked alkyne precursor through an Amadori rearrangement (Scheme D). EDCI/N-hydroxysuccinimide (NHS) promoted coupling of this amine with folic acid or the diethyl amino coumarin carboxylic acid will produce folic acid and coumarin-linked alkyne precursors, respectively (Scheme D). Aqueous acid hydrolysis of the enol ether protecting groups should provide fructose, folic acid or coumarin based alkyne precursors (Scheme D). These products and synthetic intermediates can be purified by standard chromatographic (silica or reverse phase) methods and characterized by nuclear magnetic resonance (NMR) spectroscopy and mass spectrometry (MS). Click reaction of these precursor alkynes with N₃CH₂CH₂[¹⁸F] will give the final [¹⁸F]DCP-fructose, [¹⁸F]DCP-folic acid and [¹⁸F]DCP-NEt₂-coumarin labeled compounds. These sequences yield triazole products that possess UV absorbance assisting in their identification and high-performance liquid chromatography (HPLC) purification. The precursors with protected 1,3-dicarbonyl group (before step 3 in Scheme D) will serve as non-reactive controls to tease out the contribution of cellular uptake from intracellular protein sulfenylation chemistry. Non-radioactive versions of each of these compounds can also be prepared using identical strategies but with non-radioactive fluoride ion and these controls will be characterized by both NMR spectroscopy (hydrogen, carbon and fluorine) and MS. The synthesis routes for the DCP derivatives provide high flexibility in terms of coupling order and the variation of components at each step to alter water solubility or other physical parameters.

Generation of [¹⁸F] PET Radiotracers: [¹⁸F-]-fluoride will be produced via the ¹⁸O(p,n) 18F reaction by proton bombardment (20 MeV, 16 μA) of a circulating [¹⁸O] water target at the Wake Forest PET Center Cyclotron facility on a GE PETtrace-800 cyclotron. The radioactive [¹⁸F-] will be transferred into a pyrex screw cap tube and azeotropically dried at 110° C. under a nitrogen stream using acetonitrile. [¹⁸F] PET compounds will be synthesized using copper(I) catalyzed Click reaction with the corresponding alkyne attached tosyl precursor. Reaction is carried out using a mixture of copper sulfate and sodium ascorbate as the Cu(I) source, followed by a reversed phase HPLC purification and solid phase extraction. Quality control will be performed with non-radioactive standards for authentication of the radiolabeled product and specific activity calculations.

Once prepared, the non-radioactive versions of these compounds will be evaluated for their ability to react with small molecule sulfenic acids.

The foregoing is illustrative of the present invention and is not to be construed as limiting thereof. The invention is defined by the following claims, with equivalents of the claims to be included therein.

We claim:

1. A method of synthesizing a compound of Formula I: 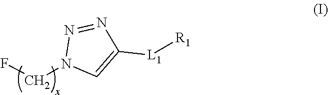
wherein:
F is fluoro;
x is an integer of from 1 to 10;
L₁ is a linker; and
R₁ is a group that is reactive with sulfenylated proteins, comprising:
A) reacting a compound of formula A: N₃—(CH₂)ₓ₊₁—Z, wherein Z is a leaving group, and x is as defined above, with fluorine, to form a compound: N₃—(CH₂)ₓ₊₁—F;
providing an alkyne-containing compound of formula B: 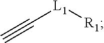
and then
reacting the compound N₃—(CH₂)ₓ₊₁—F with the alkyne-containing compound of formula B, said reacting carried out by a copper-catalyzed Click reaction, or
B) reacting a compound of formula A: N₃—(CH₂)ₓ₊₁—Z, wherein Z is a leaving group, and x is as defined above, with an alkyne-containing compound of formula B: 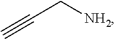
said reacting carried out by a copper-catalyzed Click reaction, to form a compound C: 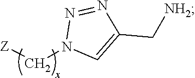
and then
adding L1-R1 to the compound to form a compound D (e.g., through amide bond formation of activated carboxylic acids): 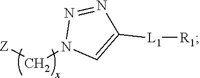
and then
reacting the compound D with fluorine to displace Z with the fluorine, to thereby synthesize said compound of Formula I.

2. A method of labeling tissues in a subject, comprising:
administering a compound of Formula I: 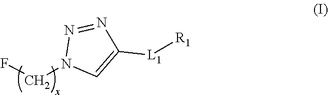
wherein:
F is fluoro;
x is an integer of from 1 to 10;
L₁ is a linker; and
R₁ is a group that is reactive with sulfenylated proteins,
to said subject, and then
performing a PET scan on the subject, wherein the PET scan detects the presence or absence of binding of said compound to said tissues, the presence of binding indicating the presence of sulfenylated proteins in said tissues.

3. The method of claim 2, wherein the labeling is carried out during cancer imaging, neuroimaging, cardiology imaging, infectious disease imaging, or imaging measuring musculoskeletal activity.

4. The method of claim 2, wherein said tissues comprise cancerous tissues.

5. The method of claim 2, wherein said administering is carried out by parenteral administration.

6. A method for determining whether a cancerous tissue has an increased likelihood of responding to radiation treatment, comprising:
administering a compound of Formula I: 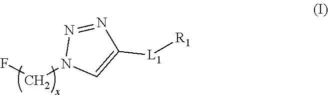
wherein:
F is fluoro;
x is an integer of from 1 to 10;
L₁ is a linker; and
R₁ is a group that is reactive with sulfenylated proteins,
to said subject, and then
performing a PET scan on the subject, wherein the PET scan detects the presence or absence of binding of said compound to said cancerous tissue, the presence of binding indicating an increased likelihood of response of said cancerous tissue to radiation treatment.

7. The method of claim 6, wherein L₁ comprises a C1-C10 alkyl, a ketone, an amide, an ester, a carbamate, a urea, an ether, a carbonate, or a combination of two or more thereof.

8. The method of claim 6, wherein R₁ comprises a 1,3 dicarbonyl group or a bicyclononyne.

9. The method of claim 6, wherein R₁ is selected from the group consisting of: 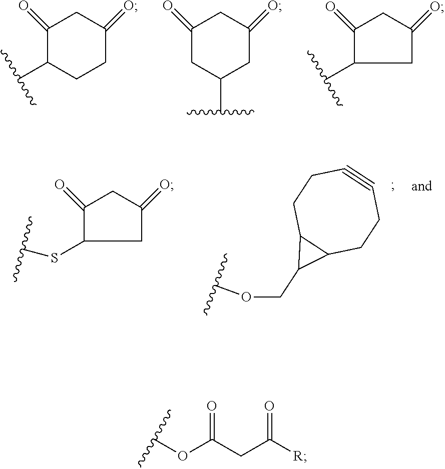
or wherein R₁ is selected from the group consisting of: 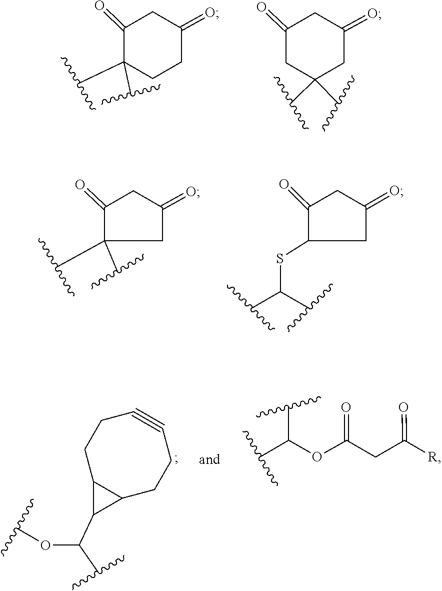
wherein R is an alkyl, ester or amide group.

10. The method of claim 6, wherein said compound is selected from the group consisting of: 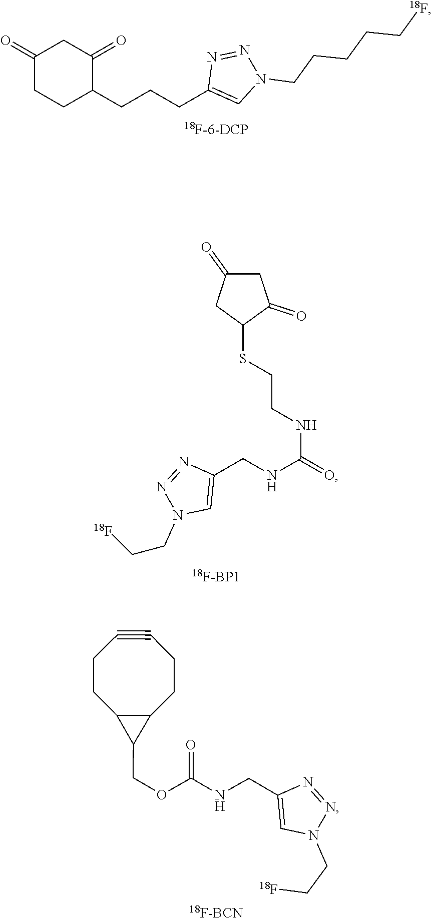
and pharmaceutically acceptable salts thereof.

11. The method of claim 6, wherein said compound is: 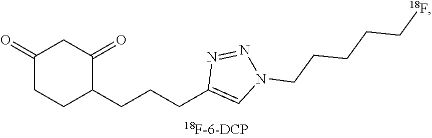
or a pharmaceutically acceptable salt thereof.

12. A method of treatment for a cancer in a subject in need thereof, comprising administering to the subject a treatment-effective amount of a compound: L₁-R₁, or L₁-R₁-L₂-R₂,
wherein:
L₁ and L₂ are each independently present or absent, and when present is independently a C1-C10 alkyl, a ketone, an amide, an ester, a carbamate, a urea, an ether, a carbonate, or a combination of two or more thereof;
R₁ is a group that is reactive with sulfenylated proteins; and
R₂ is a cellular directing group,
or a pharmaceutically acceptable salt thereof.

13. The method of claim 12, wherein the treatment-effective amount is from 1, 2, 5 or 10 to 20, 30, 40 or 50 mg per kg; or from 0.08, 0.16, 0.4, or 0.81 to 1.6, 2.4, 3.2 or 4 mg/kg.

14. The method of claim 12, wherein the administering is carried out by parenteral administration.

15. The method of claim 12, wherein the method further comprises administering radiation therapy to the subject.

16. The method of claim 12, wherein the cancer is determined to be resistant to radiation treatment or treatment targeting receptor tyrosine kinases.

17. The method of claim 16, wherein the cancer is determined to be resistant to treatment targeting epidermal growth factor receptor (EGFR).

18. The method of claim 16, wherein the cancer is determined to be resistant to treatment with erlotinib, imatinib, or afatinib.
